(19) United States
(12) Patent Application Publication
(10) Pub. No.: US 2011/0046213 A1
(43) Pub. Date: Feb. 24, 2011

(54) Production of Delta 9 Tetrahydrocannabinol

(75) Inventors: Parveen Bhatarah, Herts (GB); Derek McHattie, Herts (GB); Alan Kenneth Greenwood, Herts (GB)

Correspondence Address: STERNE, KESSLER, GOLDSTEIN & FOX P.L.L.C., 1100 NEW YORK AVENUE, N.W., WASHINGTON, DC, 20005, US

(73) Assignee: Resolution Chemicals Limited

(21) Appl. No.: 12/990,387

(22) PCT Filed: May 1, 2009
(86) PCT No.: PCT/GB09/01109

§ 371 Date: Oct. 29, 2010

(30) Foreign Application Priority Data
May 1, 2008 (GB) 0807915.4
Oct. 20, 2008 (GB) 0819141.3

(51) Int. Cl. A61K 31/352; C07D 311/80; A61P 25/00; A61P 1/08

(52) U.S. Cl. 514/454; 549/390

(57) ABSTRACT

Δ9 THC is obtained by extracting Δ9 THC and Δ9 THC carboxylic acid from plant material using a non-polar solvent and decarboxylating the Δ9 THC acid into Δ9 THC in the same solvent, without a solvent swap, in the presence of aqueous base. The Δ9 THC is then washed to remove inorganic impurities, still in the same original solvent.

### FIELD

The present invention relates to the production of Δ9 tetrahydrocannabinol (Δ9 THC), in particular to methods of its extraction from plant material and also to compositions and pharmaceutical compositions containing the extracted Δ9 THC.

### BACKGROUND

Cannabinoids are a family of naturally occurring C₂₁ terpenophenolic compounds uniquely produced in cannabis. Marijuana usually refers to a mixture of leaves and flowering heads of the pistillate plant of Cannabis sativa from which tetrahydrocannabinols (THCs) are isolated. THCs contain two main isomeric forms, depending on the position of the double bond. The position of the double bond and the stereochemistry of these THCs have been confirmed by nuclear magnetic resonance and X-ray structure.

THCs have been used as psychomimetic agents for many years with the main psychomimetic activity being attributed to Δ9-THC (20 times greater than Δ8-THC). Δ9-THC is marketed as Marinol™ and is prescribed for patients suffering from severe nausea and vomiting associated with cancer chemotherapy.

The major cannabinoids present in cannabis other than Δ9-THC and Δ8-THC are cannabinol, cannabidiol and Δ9-THC carboxylic acid which exists in two forms depending on the position of the carboxylate group. Cannabidiol may be present in cannabis in large amounts but has little activity.

The major component of cannabis is Δ9-THC carboxylic acid which exists as two isomeric forms, THCA-A and THCA-B, both of which are psychomimetically inactive. It can be converted into the predominately active constituent Δ9-THC, slowly on storage and rapidly on exposure to heat (e.g. when smoked). In fresh, dried marijuana, 95% of cannabinoids are present as THCA-A. Only THCA-A can be readily decarboxylated to Δ9-THC due to the presence of hydrogen bonding.

It is known to extract active ingredients from cannabis plant material using ethanol or a mixture of ethanol and water. The extract typically contains large amounts of Δ9-THC and Δ9-THC carboxylic acid, though accompanied by plant material which is converted to undesirable tar during later processing. To remove inorganic components from the extract a solvent swap is needed.

An alternative method of extracting Δ9 THC is also known, wherein Δ9 THC and Δ9 THC carboxylic acid are extracted from cannabis plant material into heptane. The heptane fraction extract obtained contains a mixture of cannabinoids, the main component being Δ9-tetrahydrocannabinol carboxylic acid (Δ9-THC acid). The Δ9-THC acid is extracted as its sodium salt into a dilute sodium chloride/sodium hydroxide solution, a step which removes some contaminants but also leaves behind the Δ9 THC. The salt is subsequently extracted into isopropyl ether (IPE). The Δ9-THC acid sodium salt in IPE is washed with a 2% w/v aqueous sodium hydroxide/sodium chloride solution, then acidified (pH<3) with dilute hydrochloric acid. The Δ9-THC acid solution is treated by passing through a florisil bed, to remove plant material, which is insoluble in IPE. Acidification of the Δ9-THC acid sodium salt is required prior to florisil treatment because salt will not pass through the bed. The Δ9-THC acid solution in IPE is then decarboxylated by refluxing the solution in the presence of 22% aqueous sodium hydroxide solution. The Δ9 THC product is highly purified.

Other known processes are described in WO 03/061563, US 2007/093665 and WO 2006/133941.

A number of difficulties exist in known extraction and purification processes.

The existing method described in detail above relies upon three separate solvent swaps in order to successfully remove impurities. This method is efficient in that a highly pure product may be obtained, but is as a result of the solvent swaps complex, time-consuming and not optimised for scale-up.

The USP specification for pharmaceutical compositions containing Δ9 THC, referred to as dronabinol, indicates a maximum contaminant level of cannabinoids. The step of extracting active ingredients from cannabis also extracts a number of impurities which are difficult to remove from the finished product. Despite the problems mentioned immediately above, it is accepted that a large number of solvent swaps and/or extraction steps in conjunction with chromatography are required to reduce the number of impurities, in order to meet the USP requirements.

It is generally desirable to scale-up the process and/or to improve the quantity and quality of the yield. Whilst methods with fewer solvents are known they are not suitable for large scale operation.

It is therefore an object of the present invention to provide an alternative method for production of Δ9 THC that ameliorates the difficulties in the art. An object of a specific embodiment of the invention is to provide a production method with increased yield and/or decreased impurities in the final product. A further object of a specific embodiment of the invention is to provide an improved production method with fewer and/or simpler steps to the final product, with higher yield and being suitable for use on a large scale.

### SUMMARY OF THE INVENTION

Accordingly, the present invention provides a method of production of Δ9 THC comprising extracting Δ9 THC and Δ9 THC carboxylic acid from plant material using a solvent and decarboxylating the Δ9 THC acid into Δ9 THC in the same solvent.

A further method of the invention comprises extracting Δ9 THC and Δ9 THC carboxylic acid from plant material using a solvent and decarboxylating the Δ9 THC acid into Δ9 THC, wherein the solvent is not swapped between extraction and decarboxylation.

In a second aspect of the invention there is provided a solution of Δ9 THC in a non-polar solvent comprising a straight or branched C₅-C₉ alkane, or mixtures thereof, wherein the solution is substantially free from Δ9 THC carboxylic acid.

In a specific embodiment of the invention there is provided a solution of Δ9 THC in heptane, wherein the solution comprises Δ9 THC carboxylic acid and the ratio of Δ9 THC to Δ9 THC carboxylic acid is at least 9:1.

### DETAILED DESCRIPTION OF THE INVENTION

In a first aspect of the invention there is provided a method of production of Δ9 THC comprising extracting Δ9 THC and Δ9 THC carboxylic acid from plant material using a solvent and decarboxylating the Δ9 THC acid into Δ9 THC in the same solvent. Thus, in typical operation of the invention, the solvent is not swapped between extraction and decarboxylation.

A particular method of production of Δ9 THC comprises:—
(i) extracting Δ9 THC and Δ9 THC carboxylic acid from plant material using a non-polar solvent, to yield a solution containing Δ9 THC and Δ9 THC carboxylic acid; and
(ii) decarboxylating the Δ9 THC carboxylic acid into Δ9 THC in the same non-polar solvent in the presence of aqueous base.

Another particular method of production of Δ9 THC comprises:—
(i) extracting Δ9 THC carboxylic acid from plant material using a non-polar solvent, to yield a solution containing Δ9 THC carboxylic acid; and
(ii) in the presence of aqueous base, heating the solution and thereby decarboxylating the Δ9 THC carboxylic acid into Δ9 THC in the same non-polar solvent.

The extraction of Δ9 THC carboxylic acid, or of Δ9 THC and Δ9 THC carboxylic acid, from plant material and the subsequent decarboxylation of Δ9 THC acid into Δ9 THC are carried out in the same solvent. Thus, a solvent, which can be a mixture of solvents, is selected for the extraction step and is used throughout the process up to and including the decarboxylation of Δ9 THC acid into Δ9 THC. Separate extracts may be combined and the solution of Δ9 THC and/or Δ9 THC acid may be concentrated or diluted at different stages but the solvent system does not change.

In known methods some of the extracted Δ9 THC is discarded at an early processing stage, effectively sacrificed as part of the removal of contaminants. The present invention uses a single solvent and does not discard Δ9 THC in this way, thus increasing the Δ9 THC available to contribute to the overall yield at the end of the extraction.

The solvent is suitably a non-polar solvent or a mixture of non-polar solvents, with alkanes as described below being particularly suitable as solvent components. A number of non-polar solvents are suitable for the extraction, and these solvents include straight and branched C₅-C₉ alkanes, in particular pentane, hexane, heptane, octane, and nonane, other petrol fractions, other solvents immiscible with water and mixtures of the aforementioned. The alkanes and mixtures of the alkanes are preferred. In an example of the invention set out in detail below, particularly good results have been obtained using heptane.

The solvent is preferably degassed before use. In an example of the invention set out in detail below, particularly good results have been obtained when the solvent is degassed with nitrogen before use. Degassing the solvents and other solutions used in the production process tends to lead to fewer impurities in the Δ9 THC extract.

The solvent solutions are generally easy to handle throughout the production process. A specific advantage of using heptane is that it facilitates the extraction and decarboxylation processes.

As described in more detail in examples below, decarboxylation of Δ9 THC carboxylic acid takes place in solution, not from a Δ9 THC carboxylic acid-containing residue. The method is thus suitable for use on a large scale.

As further described in more detail in examples below, the method preferably takes place in the presence of aqueous base. The base preferably comprises an alkali metal oxide or hydroxide, for example sodium hydroxide, though choice of base is not thought to be critical.

In embodiments of the invention, the method comprises extracting the Δ9 THC and Δ9 THC carboxylic acid from plant material using a 2-phase extraction process comprising:

a) combining the plant material and a solvent to form a mixture; b) extracting the Δ9 THC and Δ9 THC carboxylic acid; c) separating the mixture into (1) a first extract and (2) plant material; d) combining the plant material from (c) and further solvent to form a mixture; e) extracting the Δ9 THC and Δ9 THC carboxylic acid; f) separating the mixture into (1) a second extract and (2) plant material; and g) combining the first and second extracts.

A preferred method of the invention comprises extracting the Δ9 THC and Δ9 THC carboxylic acid from plant material using a 3-phase extraction process comprising: a) combining the plant material and a solvent to form a mixture; b) extracting the Δ9 THC and Δ9 THC carboxylic acid; c) separating the mixture into (1) a first extract and (2) plant material; d) combining the plant material from (c) and further solvent to form a mixture; e) extracting the Δ9 THC and Δ9 THC carboxylic acid; f) separating the mixture into (1) a second extract and (2) plant material; g) combining the plant material from (f) and further solvent to form a mixture; h) extracting the Δ9 THC and Δ9 THC carboxylic acid; i) separating the mixture of (g) into (1) a third extract and (2) plant material; and j) combining the first, second and third extracts.

In the 2- and 3-phase methods, after each extraction step the extracted mixture (containing solvent, Δ9 THC, Δ9 THC carboxylic acid and plant material) is separated into at least (i) an extract containing Δ9 THC and Δ9 THC carboxylic acid, and (ii) plant material, and then the plant material is passed to a further extraction step using further fresh solvent. Hence, increased extraction from the plant material can be achieved. Preferably the plant material extracts are combined and concentrated before decarboxylation.

During decarboxylation the Δ9 THC acid is typically heated under reflux under a nitrogen atmosphere and in specific embodiments of the invention the reaction is subsequently stopped and the mixture is cooled to 25 to 30° C. and degassed purified water added.

As the Δ9 THC carboxylic acid is in solution the operating temperature is generally limited by the solvent boiling point. The reflux temperature is preferably below 105° C., more preferably below 100° C. In specific examples below, the method is carried out using heptane as solvent and the decarboxylation temperature is below 100° C., generally around the boiling point of heptane, i.e. around 98-99° C. Avoiding excessive temperature during this step helps avoids conditions which risk degradation to the Δ9 THC.

After decarboxylation the Δ9 THC extract can be washed to remove organic impurities, e.g. washed with aqueous solutions or water, again without change of solvent. The product of such a washing step is referred to as “isolated Δ9 THC”.

The invention additionally provides solution of Δ9 THC in a non-polar solvent comprising a straight or branched C₅-C₉ alkane, or mixtures thereof, wherein the solution is substantially free from Δ9 THC carboxylic acid. In particular embodiments of the invention the solvent comprises pentane, hexane, heptane, octane, and nonane, or mixtures thereof, other petrol fractions and other solvents immiscible with water. In an example of the invention set out in detail below, particularly good results have been obtained using heptane. In a preferred aspect of the invention the solution comprises 10% or less, preferably 5% or less, more preferably 1% or less Δ9 THC carboxylic acid w/w with respect to Δ9 THC. The Δ9 THC is further preferably in washed or isolated form, that is to say substantially free of inorganic impurities.

An alternative embodiment of the invention provides a solution of Δ9 THC in heptane, wherein the solution comprises Δ9 THC carboxylic acid and the ratio of Δ9 THC to Δ9 THC carboxylic acid is at least 9:1, typically at least 25:1 and in preferred embodiments of the invention the ratio of Δ9 THC to Δ9 THC carboxylic acid is at least 50:1 or at least 100:1. The solution of Δ9 THC in heptanes is preferably in washed or isolated form, that is to say substantially free of inorganic impurities.

The present invention has the advantage that it can provide a more complete extraction of Δ9 THC. In the prior art it is known to discard some Δ9 THC during the initial phase of extraction as it does not convert into a sodium salt. Δ9 THC carboxylic acid is thus preferentially extracted in one of the steps. In contrast, in the method of the invention, no Δ9 THC is discarded in this way during the extraction process, leading to an increased yield of Δ9 THC.

The method of the present invention requires fewer manipulations than that used in the prior art. The process can hence be faster and easier to scale-up, with reduced waste.

Only one solvent composition is required and this is used for all steps from extraction to decarboxylation so no solvent swap is needed.

Following decarboxylation of the Δ9 THC extract it is generally necessary to further purify and isolate the Δ9 THC. This is usually carried out by passing the extract through a charcoal column, collecting the fractions containing Δ9 THC, combining and concentrating these fractions in a solvent and purifying the product by reverse phase chromatography. The final product is then concentrated and the solvent is evaporated.

A further advantage of the present invention is that the Δ9 THC must be in a particular solvent in order to carry out charcoal treatment. Following the invention, the product of decarboxylation can be applied directly to the columns for purification. Alternatively, the solution of Δ9 THC may be mixed with another solvent such as tert-butyl methyl ether (TBME) or swapped into a solvent such as TBME in a single solvent swap step. In an example of the invention set out in detail below, particularly good results were obtained when the product was swapped into and loaded onto a charcoal column as a solution in TBME. The product of the invention is thus suitable for subsequent processing steps with reduced solvent swap steps—previous methods yielded e.g. Δ9 THC in iso-propyl ether which needs two solvent swaps before it can be loaded onto the column.

Another advantage is that when using a non-polar solvent, such as heptane, inorganic impurities can be removed by washing; when e.g. ethanol is used this is not possible and a solvent swap must occur to remove the inorganic impurities. Thus the present invention enables extraction and isolation of Δ9 THC, in that a crude Δ9 THC from which inorganic impurities have been removed is isolated, in a single solvent.

### EXAMPLE 1

### 1. Extraction

Every part of this procedure was performed under a nitrogen atmosphere and ambered glassware was used at all times.

Cannabis plant material (1 kg) was shredded for 2 minutes using a food processor.

### First Extraction

A nitrogen purged ambered reaction vessel was charged with 10 volumes of n-heptane. The n-heptane was degassed for 5-10 minutes with nitrogen and the shredded plant material was added. The mixture of n-heptane and shredded plant material was stirred under a nitrogen atmosphere for 4-4.5 hours at 20-25° C. The plant material was then removed by filtering the mixture through a GF/F filter pad.

### Second Extraction

A nitrogen purged ambered reaction vessel was charged with 5 volumes of n-heptane and the filtrate from the first extraction was added. The mixture of n-heptane and shredded plant material was stirred under a nitrogen atmosphere for 1 hour and the suspension was filtered through a GF/F filter.

### Third Extraction

A nitrogen purged ambered reaction vessel was charged with 5 volumes of n-heptane and the filtrate from the second extraction was added. The mixture of n-heptane and shredded plant material was stirred under a nitrogen atmosphere for 4 hours and the suspension was filtered through a GF/F filter.

The extracts were then combined and concentrated at 35-40° C. under reduced pressure in ambered glassware to 7.5 volumes with respect to the input weight of the shredded plant material.

The vacuum was released under nitrogen and Celite® was added to the reaction vessel. The suspension was stirred for 30 minutes and filtered through hardened 54 filter paper. The reaction flask was rinsed with n-heptane which was in turn used to wash the generated Celite® filter pad.

The Celite® was pulled dry under a blanket of nitrogen until no further filtrate was removed. The filtrate was then concentrated at 35-40° C. until the volume was 2.4 with respect to the shredded plant material input.

### 2. Decarboxylation

Every part of this procedure was performed under a nitrogen atmosphere and ambered glassware was used at all times.

Pearl sodium hydroxide (170.4 g) was carefully added to a stirred solution of purified water (604.0 g) over a period of 10-20 minutes.

A nitrogen purged ambered reaction vessel was charged with the n-heptane solution of THC filtrate (1.596 Kg) and the 22% w/w sodium hydroxide was added while stirring at 20-25° C.

The reaction mixture was then heated under reflux under a nitrogen atmosphere for 2.5 hours.

The reaction mixture was cooled to 25-30° C. and 1.6 volumes of degassed purified water were added. The mixture was stirred for 15 minutes and the layers were allowed to separate for a further 5 minutes. The aqueous layer was removed. Celite® was added to the upper organic phase and the suspension was stirred for 20 minutes before being filtered through a Whatman® 54 filter paper under a nitrogen atmosphere. The reaction flask was rinsed with degassed n-heptane and this was used to rinse the generated Celite® filter pad. The Celite® filter pad was pulled dry until no more filtrate was removed from the filtered pad and any remaining water in the filtrate was separated.

The organic layer was then concentrated at 35-40° C. under reduced pressure to a thick oil.

### 3. Purification

All parts of the following procedure were performed under a dry nitrogen atmosphere and all process streams were protected from light.

### First Filtration The heptane solution was concentrated to an oil at 37-39° C./90-72 mbar until no further heptane was collected by distillation.

3 volumes of methyl tert-butyl ether (MTBE) were added to the solution (based on the assayed weight of the plant extract).

The solution was then charcoal filtered and eluted with MTBE using nitrogen pressure and fractions were collected in nitrogen purged containers containing methanol.

The fractions were sampled for gradient HPLC analysis and fractions that met the specification were combined and further concentrated to 1 volume in methanol.

### Second Filtration

The methanol solution was further purified using a 150 C18 reverse phase Biotage® cartridge. The cartridge was first eluted with 50% volume methanol/water (2 column volumes) and was then eluted with 75/25 v/v methanol/water and fractions were collected in nitrogen purged containers. The fractions were tested using TLC stained with Fast Blue and those fractions showing a positive colour test were examined by gradient HPLC. Those fractions meeting the HPLC limits were then combined.

### Isolation

All parts of the following procedure were performed under a dry argon atmosphere and all process streams were protected from light.

The combined fractions (a methanol/water solution) were concentrated at 37-39° C. under vacuum until 85-90% of the methanol was collected. The resulting opaque mixture was then extracted with MTBE at 20-25° C. The extract was stirred with magnesium sulphate and filtered. Ethanol was added to the filtrate and the solution was concentrated at 37-39° C./240-220 mbar and then to 30 mbar to produce an oil. The oil was held at 37-47° C. and a flow of argon was passed into the oil and the system was evacuated to less than 10 mbar until the solvent content was less than 5000 ppm.

The final isolated pure product was stored at less than −10° C. under argon.

The invention thus provides methods for the production of Δ9 THC.

1. A method of production of Δ9 THC comprising:—
(i) extracting Δ9 THC and Δ9 THC carboxylic acid from plant material using a non-polar solvent, to yield a solution containing Δ9 THC and Δ9 THC carboxylic acid; and
(ii) decarboxylating the Δ9 THC carboxylic acid into Δ9 THC in the same non-polar solvent in the presence of aqueous base.

2. (canceled)

3. The method of claim 1, comprising decarboxylating the Δ9 THC carboxylic acid at a temperature of 105° C. or below.

4. The method of claim 1, wherein the solvent comprises a straight or branched C₅-C₉ alkane.

5. The method of claim 1, wherein the solvent consists substantially of heptane.

6-8. (canceled)

9. The method of claim 1, wherein the solvent is degassed before use.

10. (canceled)

11. The method of claim 1, wherein during decarboxylation the Δ9 THC acid is heated under reflux under a nitrogen atmosphere.

12. The method of claim 1, further comprising washing the solution of Δ9 THC with an aqueous solution to remove inorganic impurities.

13. A method of production of Δ9 THC comprising extracting Δ9 THC and Δ9 THC carboxylic acid from plant material using a non-polar solvent to yield a solution which contains Δ9 THC carboxylic acid, and heating the solution in the presence of aqueous base so as to decarboxylate the Δ9 THC acid into Δ9 THC, wherein the solvent is not swapped between extraction and decarboxylation.

14. The method of claim 13, wherein the solvent comprises a straight or branched C₅ -C₉ alkane, or mixtures thereof.

15. The method of claim 14, wherein the solvent comprises heptane.

16. The method of claim 14, wherein the solvent consists substantially of heptane.

17-18. (canceled)

19. The method of claim 13, further comprising washing the solution of Δ9 THC with an aqueous solution to remove inorganic impurities.

20. A solution of Δ9 THC in a non-polar solvent comprising a straight or branched C₅-C₉ alkane, or mixtures thereof, wherein the solution is substantially free from Δ9 THC carboxylic acid.

21. The solution of claim 20, wherein the solvent comprises heptane.

22. The solution of claim 20, substantially free of inorganic impurities.

23. The solution of claim 20, wherein the solution comprises 5% or less Δ9 THC carboxylic acid w/w with respect to Δ9 THC.

24. A solution of Δ9 THC in heptane, wherein the solution comprises Δ9 THC carboxylic acid and the ratio of Δ9 THC to Δ9 THC carboxylic acid is at least 9:1.

25-28. (canceled)

29. A method of production of Δ9 THC comprising:—
(i) extracting Δ9 THC carboxylic acid from plant material using a non-polar solvent, to yield a solution containing Δ9 THC carboxylic acid; and
(ii) in the presence of aqueous base, heating the solution and thereby decarboxylating the Δ9 THC carboxylic acid into Δ9 THC in the same non-polar solvent.

30. The method of claim 29, comprising decarboxylating the Δ9 THC carboxylic acid at a temperature of 105° C. or below.

31. The method of claim 29, wherein the solvent comprises a straight or branched C₅-C₉ alkane.

32. The method of claim 29, wherein the solvent consists substantially of heptane.
